(19) United States
(12) Patent Application Publication
(10) Pub. No.: US 2008/0207950 A1
(43) Pub. Date: Aug. 28, 2008

(54) Enantioselective Synthesis of a Sterically Hindered Amine

(75) Inventors: Ulrich Berens, Binzen (DE); Christophe Malan, Neuchatel (CH); Hans Jurg Kirner, Pratteln (CH)

Correspondence Address: JoAnn Villamizar;Ciba Corporation/Patent Department, 540 White Plains Road, P.O. Box 2005, Tarrytown, NY, 10591, US

(73) Assignee: CIBA SPECIALTY CHEMICALS CORP., TARRYTOWN, NY (US)

(21) Appl. No.: 11/792,744

(22) PCT Filed: Dec. 12, 2005
(86) PCT No.: PCT/EP05/56678

§ 371 Date: Jun. 12, 2007

(30) Foreign Application Priority Data
Dec. 22, 2004 (EP) 04106820.6

(51) Int. Cl. C07C 209/00; C07C 209/24; C07C 231/00

(52) U.S. Cl. 564/219; 564/305; 564/338

(57) ABSTRACT

Compounds of the formula (I) wherein R is phenyl, or phenyl substituted by Cl, Br, C₁-C₄alkyl or CF₃; R₁ is H or methyl or ethyl; R₂ is H or methyl or acyl; R₃ is H or methyl; R′₄ is —CH₃ or ═CH₂; may be obtained in high enantiopurity by hydrogenation of a compound of the formula (II) wherein R and R₃ are as in formula (I); A is acyl; and R₄ is —CH₃ or ═CH₂; in the presence of a chiral Rhodium or Ruthenium catalyst. Residues R₁ as methyl or ethyl and/or R₂ as H or methyl may subsequently be introduced without racemization by deacylation and optional alkylation.

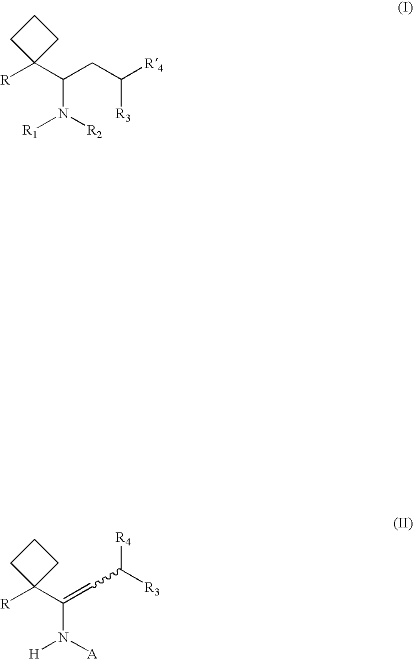

### BACKGROUND TO THE INVENTION

Currently, racemic Sibutramine 1 is licensed for the treatment of obesity. On absorption, the drug is rapidly metabolized to give the primary metabolites des-methylsibutramine 2 and di-desmethyl-sibutramine 3. Preliminary preclinical studies suggest that the potent serotonin, norepinephrine, and dopamine re-uptake inhibitor (R)-2 might be useful for the treatment of CNS disorders (WO 00/10551). Also, the enantiomers of 3 have been claimed for the treatment of depression and related disorders (WO 94/00047 and WO 94/00114).

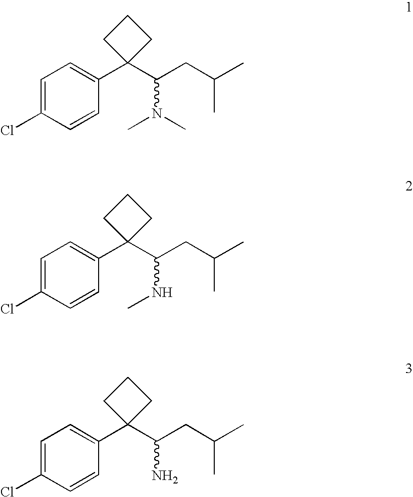

An efficient route to give one of the compounds 1-3 with high enantiopurity will allow accessing the remaining compounds either via methylation or de-methylation steps. Clearly, an efficient route to give 3 in high enantiopurity as either enantiomer is most desirable, as the mono- or di-methylation of this compound to give 2 or 1 is a much simpler transformation compared with de-methylation chemistry. The present invention provides such a route to 3, and thus also an efficient access to either 1 or 2 by reductive amination or selective mono-methylation of 3.

The resolution of racemic 1 with a chiral acid and the de-methylation of the resolved 1 with diethyl-azodicarboxylate (DEAD) to give enantiopure 2 is known (Tetrahedron: Asymmetry 1999, 10, 4477). However, DEAD can decompose violently, which has ruled out this approach for the production of commercial quantities of 2. Therefore, a procedure for the resolution of 2 with chiral acids was developed (Tetrahedron: Asymmetry 2002, 13, 107). In such a resolution process half of the starting material is lost, unless it can be re-utilized e.g. by a racemisation process. As also the efficiency of the resolution was low, a catalytic asymmetric synthesis of (R)-2 by the enantioselective addition of isobutyl lithium to imine 5 was developed. This approach is potentially very attractive, but even with the best identified catalyst a product with only 40% ee could be obtained. This required a subsequent resolution in order to upgrade the ee to enantiopurity (Tetrahedron Lett. 2002, 43, 2331):

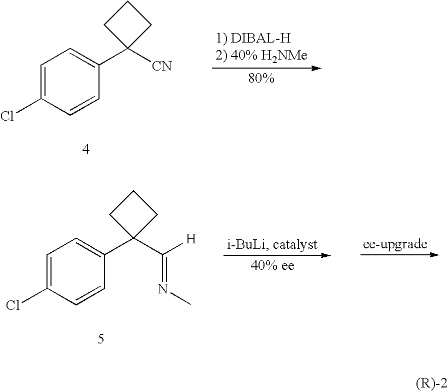

This drawback led to the development of yet another route, where the key step is the addition of isobutyl lithium to the chiral sulfinimide 6. Hydrolysis of the resulting product gives (R)-3 with excellent yield and enantiopurity (Org. Lett. 2002, 4, 4025). However, this addition is a low temperature reaction (optimum conditions at −78° C.), and the required auxiliary, which has to be prepared in three steps is destroyed and thus lost in the workup, which significantly impairs the economy of the process:

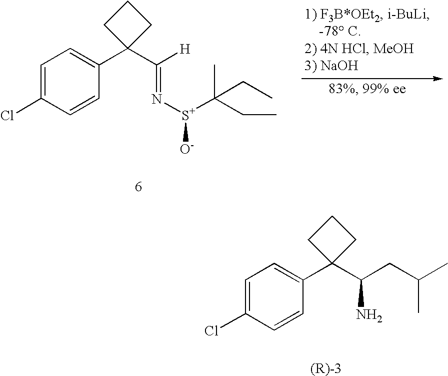

Stereoselective hydrogenation of some dienamides with cationic Rh(I)-catalysts to provide the γ,δ-unsaturated amide has been reported in J. Am. Chem. Soc. 1998, 120, 657.

### DESCRIPTION OF THE INVENTION

The present invention describes an efficient route to obtain 3 in high enantiopurity as either enantiomer, as well as its conversion into 1 or 2.

The generation of the stereogenic carbon in a precursor to 3 from a prochiral carbon centre in a suitable substrate via asymmetric hydrogenation in high ee would circumvent the formation of the undesired enantiomer and thus its loss, respectively the need to utilize this material for example by a racemisation/resolution process.

Substrates such as 8 appear to be suitable substrate candidates, as it is known that certain enamides can be hydrogenated very efficiently with cationic Rh(I)-complexes of DuPHOS-type ligands; these enamides may be obtained by the reduction of a suitable oxime with iron in the presence of acetic acid and acetic anhydride in analogy to methods described in J. Org. Chem. 1998, 63, 6084:

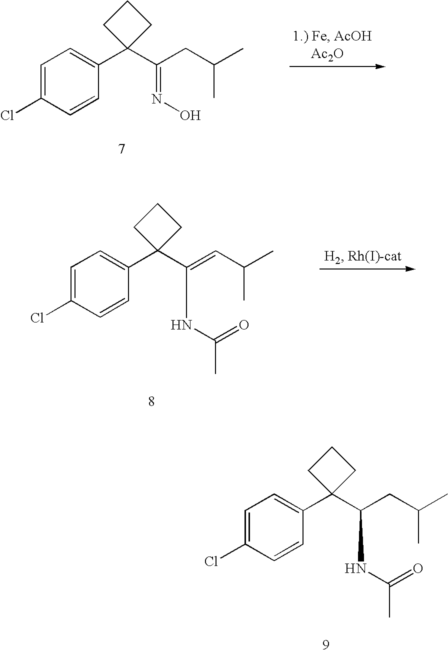

However, the reduction of oxime 7 according to the published protocol gave 8 in only 32% yield. An alternative approach to enamides such as 8 involves the reaction of Grignard reagents with nitriles. Such an approach usually requires chromatographic purification of the products, and the yields are low (see, for example, J. Org. Chem. 1998, 63, 6084 and Bull Soc. Chim Fr. 1965, 1454).

It was thus surprising, that the reaction of isobutyl magnesium halide with nitrile 4 and treatment of the reaction mixture with acetic anhydride in-situ gives enamide 8 in high yield (such as 62% in present example 1b), though three additional steps are required for the synthesis of 7 alone: (i) quench of the Grignard addition reaction with water, (ii) imine hydrolysis with hydrochloric acid to the ketone, and (iii) formation of the oxime 7.

More preferably, nitrile 4 is reacted with methallyl magnesium halide to yield the novel dienamide 10 as the Z-stereoisomer. This may be achieved, for example, by treatment of the reaction mixture with acetic acid anhydride and a basic workup.

8 may be obtained by hydrogenation of enamide 10 with cationic Rh(I)-catalysts derived from Me-DuPHOS, Me-BPE or Et-Ferrotane ligands. Not the α,β-double bond, but the γ,δ-double bond is hydrogenated preferentially here, resulting in the formation of 8 as a substrate of very low reactivity with these catalysts.

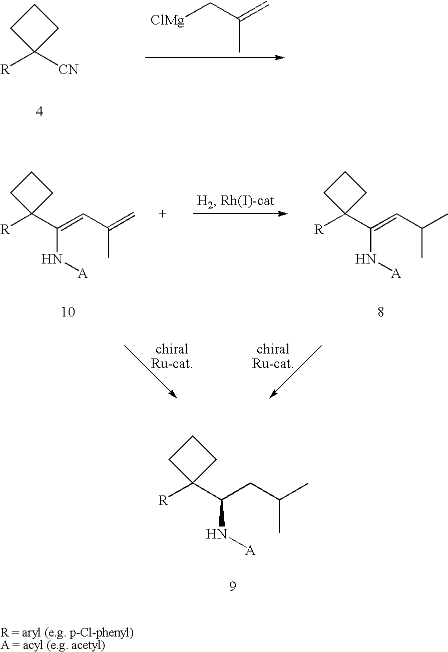

The present approach usually leads to the Z-isomers of 8 and 10 shown above. Both isomers Z and E, however, may be transformed to the desired end products 9 and, especially, 1-3 in high enantiopurity. Enamides of the below formula II, e.g. 8 or 10 shown above, are preferably in the form of the Z-isomer.

Present invention thus relates to a process for the enantioselective preparation of a compound of the formula I

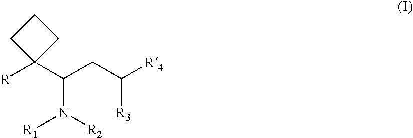

wherein
R is phenyl, or phenyl substituted by Cl, Br, C₁-C₄alkyl or CF₃;
R₁ is H or methyl or ethyl;
R₂ is H or methyl or acyl;
R₃ is H or methyl;
R′₄ is —CH₃ or ═CH₂;

characterized in that a compound of the formula II

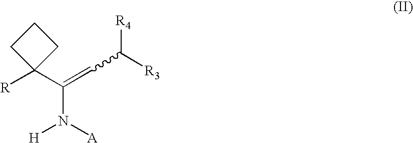

wherein R and R₃ are as in formula I; A is acyl; and R₄ is —CH₃ or ═CH₂;

is hydrogenated in the presence of a chiral Rhodium or especially Ruthenium catalyst, and a residue R₁ as methyl or ethyl and/or R₂ as H or methyl is subsequently introduced by deacylation and optional alkylation, especially methylation.

The compound of the formula

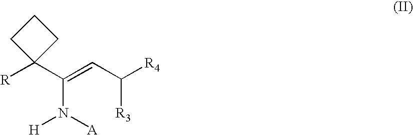

may be formulated alternatively as its tautomer of the formula II′:

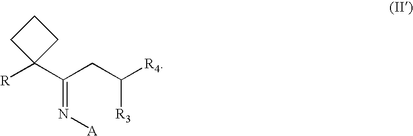

The primary product from the hydrogenation of the dienamide (e.g. 10) would be expected to be the γ,δ-unsaturated amide, which would require hydrogenation of the remaining double bond with a heterogeneous catalyst to give the product of formula I (e.g. 9). Quite surprisingly, it has been found that the remaining olefinic bond formed is also hydrogenated with a homogeneous catalyst under the reaction conditions, thus yielding the N-acyl amide of formula I.

The invention provides the instant products of the formula I (e.g. 9 and, especially, 1-3 further above, or other species of the formula I) with high enantiomeric excess (ee; high enantiomeric purity or high enantiopurity);

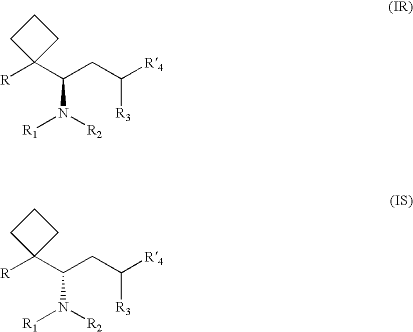

whereever used, these terms stand for e.g. an ee of 60-100%, preferably 80-100%, especially 90-100%, of the (R)- or of the (S)-enantiomer. Thus, in its most preferred embodiment the process according to the invention yields the compound of the formula I with an enantiomeric excess of 90% or more of either the (R) or the (S)-enantiomer.

The compound of formula I may be obtained in, or converted into, the form of a pharmaceutically acceptable salt and/or suitable crystalline form.

Useful acid addition salts of compounds of present invention include those with inorganic acids, such as chlorides or sulfates, or with organic acids, e.g. sulfonic or carbonic acids, such as methane sulfonates, benzoates, oxalates or acetates, where appropriate and expedient. Salts of compounds of the formula I are preferably pharmaceutically acceptable salts, while for the purposes of isolation or purification especially of the salts of other compounds mentioned above and below it is also possible to use pharmaceutically unsuitable salts, for example picrates or perchlorates. Only the pharmaceutically acceptable salts or the free compounds (optionally in the form of pharmaceutically compositions) of the compounds of formula I are used therapeutically and they are therefore preferred, e.g. benzoate, hydrochloride or hydrogen sulfate. Pharmaceutically acceptable salts are addition salts mostly known in the art, e.g. of acids like alkanecarboxylic acids (especially of C₁-C₄acids); di- or polycarboxylic and/or hydroxycarboxylic acids such as oxalic, malonic, succinic, fumaric, citric, maleic, tartaric, lactic acid, glucuronic acid and other acids derived from sugars, each of these acids in both enantiomeric forms where optically active; phosphoric, sulfuric, methylsulfonic, toluenesulfonic, benzoic acid; some preferred salts include hydrochlorides, hydrobromides, hydroiodides, benzoates, phosphates, hydrogenphosphates, sulfates, hydrogensulfates etc.

Any acyl group mentioned such as R₂ or the residue A, e.g. in the compound of formulae I and II, preferably is C₁-C₄alkanoyl or benzoyl, especially formyl or acetyl.

R₁ is preferably H or methyl.

R is preferably 4-chlorophenyl. R₂ in the present product is preferably H or methyl. R₃ and R′₄ each is preferably methyl. Preferred products of the present process are sibutramine or N-monodesmethyl sibutramine or N,N-didesmethyl sibutramine.

It has further been found that, as a crystalline solid, the amide of formula I (e.g. compound No. 9) of high enantiomeric purity, especially when reaching an ee of 92% or more, lends itself to an ee-upgrade by crystallisation. Recrystallization of the product having an already high ee (e.g. >92%, especially >96%) may yield an amide with ee of well over 99%. Suitable solvents include alcohols, ketones and ethers, such as ethanol, methanol, di-isopropyl ether etc.). This behavior, especially of compound 9, is a very useful feature of the present invention, as the possibility to upgrade the ee allows to choose more freely between a variety of hydrogenation catalysts respectively the corresponding ligands. Consequently, present invention includes a process, wherein the compound of formula I obtained, wherein R₂ is acyl, is crystallized or recrystallized; most preferably, R₂ is chosen as acetyl in such a process.

Asymmetric hydrogenation of the enamide of formula II (such as compound No. 8), especially those of the formula II wherein R₄ is ═CH₂ (such as conversion of the compound No. 10 to No. 11 and especially to No. 9) of high enantiopurity may be achieved with a catalyst of the type [Rh PP diolefine]⁺ anion, where PP stands for 2 monodentate ligands or 1 bidentate ligand, the diolefine may be norbornadiene or preferably cyclooctadiene, und the anion preferably is selected from BF₄⁻, ClO₄⁻, PF₆⁻, BARF⁻.

In order to achieve both satisfactory conversion and high ee, the asymmetric hydrogenation of the compound of formula II (e.g. of 10 and also of 8) to give the product of formula I (e.g. compound 9) preferably is accomplished with a chiral Rh- or especially with a Ru-catalyst derived from an axially chiral enantiopure ligand.

Preferred is a Ru-catalyst of the type [Ru PP X₂], where PP is a ligand of axial chirality; examples for such ligands are BINAP, BIPHEP, BIPHEMP, SEGUPHOS, BITAMP, BIBFUB, SYNPHOS, HEXAPHEMP, TetraPHEMP, TUNAPHOS; PP may alternatively be a planar chiral ligand of the phanephos backbone; X is an anion, e.g. a halogenide, a carboxylate such as an acetate etc. A comprehensive survey of such ligands can be found in Chem. Rev. 2003, 103, 3029, chapter 2.3.1 or also in Adv. Synth. Catal. 2003, 345, No. 1+2, 103.

Preferred examples for such ligands are BINAP or BIPHEMP and the like (see further below). Selection of the ligand chirality determines the chirality of the reaction product (S or R). A comprehensive survey of such ligands can be found in Chem. Rev. 2003, 103, 3029, chapter 2.3.1 or also in Adv. Synth. Catal. 2003, 345, No. 1+2, 103. Preferred is a Ruthenium catalyst containing an axially chiral or planar chiral bisphosphine ligand.

The asymmetric hydrogenation may be carried out using high ratios of substrate/catalyst (S/C), e.g. 100-100000, preferably 200-20000. In case of high S/C ratios, the efficiency of the catalyst (conversion rates at high substrate/catalyst ratios) may be greatly improved by adding a substance containing a coordinating anion as a rate enhancing additive. Preferred coordinating anions are those excluding those classified as hard bases according to R. G. Pearson, examples for such substances are protic acids, especially apart from HF, especially mineral acids including HCl, HBr, HI, or a solution thereof, carboxylic acids such as acetic acid, or salts containing a coordinating anion (especially salts composed of a hard acid and a soft base, see above) such as lithium chloride or bromide. The rate enhancing effect is especially advantageous when using the chiral Ru-catalyst such as BINAP. The acidic substance usually is added in catalytic amounts, e.g. in an amount of 0.01 to 1, especially 0.1 to 1 equivalent H⁺ per mol of substrate.

Example: full conversion at S/C=1000 after adding a small quantity of hydrochloric acid to the reaction mixture.

In a preferred process of the invention, therefore, the ratio of substrate of the formula II to chiral Ruthenium catalyst is greater than 100 and a substance containing a coordinating anion, especially a protic acid or a lithium salt, is added.

Hydrogenation is effected using methods and equipment known in the art, hydrogen pressures applied, e.g. between about 0.1 to 200 bar, are not critical, hydrogen pressure often ranges from about 1 to about 200 bar, preferred is the moderate pressure range, e.g. 5 to 100 bar.

Some of the compounds obtained according to the process of the invention are novel. Thus, present invention also pertains to a compound of the formula II

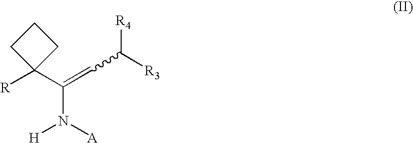

or of the formula VII

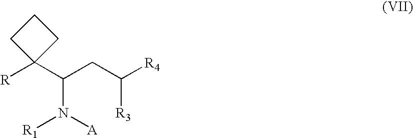

wherein R is phenyl, or phenyl substituted by Cl, Br, C₁-C₄alkyl or CF₃, and R especially is 4-chlorophenyl; R₁ is H or methyl; R₃ is H or especially methyl; R₄ is ═CH₂ or especially —CH₃; and A is C₁-C₄alkanoyl, especially formyl or acetyl. Compounds of the formula VII usually are of high enantiopurity, e.g. with an enantiomeric excess of 90% or more. The invention therefore further pertains to a composition containing a mixture of the enantiomers of the formula VII (R) and (S)

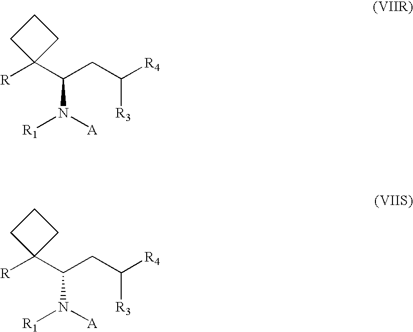

wherein R is phenyl, or phenyl substituted by Cl, Br, C₁-C₄alkyl or CF₃, and R especially is 4-chlorophenyl; R₁ is H or methyl; R₃ is H or especially methyl; R₄ is ═CH₂ or especially —CH₃; and A is C₁-C₄alkanoyl, especially formyl or acetyl; or an addition salt thereof with a pharmaceutically acceptable acid,

characterized in that the enantiomeric excess of one of said enantiomers is at least 60%, or higher as described further above.

The key educt of the present process may advantageously be obtained by reacting a suitable cyclobutyl nitrile with a Grignard reagent, e.g. of the alkyl or allyl type, followed by acylation of the amino group. Present invention therefore includes a process for the preparation of a compound of the formula II

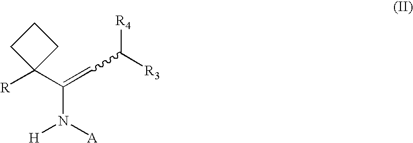

where R is phenyl, or phenyl substituted by Cl, Br, C₁-C₄alkyl or CF₃, and R especially is 4-chlorophenyl; R₃ is H or especially methyl; R₄ is —CH₃ or especially ═CH₂, and A is acyl such as C₁-C₄alkanoyl, especially acetyl; comprising the steps
i) reaction of a nitrile of the formula III

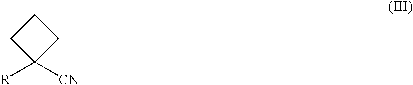

where R is as defined for formula II;

with a reagent of the formula IV

Hal-Mg—CH₂—C(R₃)(R₄) (IV)

where Hal stands for halogen, especially chloro or bromo, and R₃ and R₄ each are as defined for formula II;
ii) reaction of the metal organic intermediate with an acylating agent introducing the moiety A; and optionally
iii) conversion of a diamide formed, if so, into the compound of formula II by reaction with a suitable base.

The Grignard reagent of the formula IV is preferably selected from isobutyl magnesium chloride, isobutyl magnesium bromide, methallyl magnesium chloride or methallyl magnesium bromide. The acylating agent used in step ii) preferably is an acyl halide, or especially an anhydride, of the formula V or VI

Hal-A (V)

A-O-A (VI)

where A is as defined for formula II and Hal is as defined for formula IV.

Formula VI comprises anhydrides of one acid as well as mixed anhydride such as CH₃COOCHO (for formylation). If acid halogenides of formula V are used, these usually are C₂-C₄carboxylic acid halogenides or a benzoyl halogenide, especially chlorides. The base used in step iii) preferably is selected from alkoholates and hydroxides of alkali or alkaline earth metals such as Li, Na, K, Rb, Cs, Ba, especially from the group consisting of NaOCH₃, NaOC₂H₅, NaOC₃H₇, KOCH₃, KOC₂H₅, KOC₃H₇, LiOCH₃, LiOC₂H₅, LiOC₃H₇, NaOH, KOH, LiOH, CsOH, Ba(OH)₂.

The de-acetylation of the compound of the formula I, where R₂ is acyl, or of the compound of the formula VII (e.g. of 9 to give 3) may be accomplished under conditions known in the art, e.g. by base cleavage or preferably by acidic cleavage, e.g. by adding protic acid such as a hydrogen halide or solution thereof, sulfuric acid etc., especially hydrochloric acid such as concentrated aqueous HCl. The process using acidic conditions preferably gives the acid addition salt. Cleavage is preferably carried out at elevated temperature, e.g. 80-200° C., especially 150-200° C. or near 180° C., usually under pressure. Surprisingly, even at harsh conditions during the cleavage, the ee of the starting material is retained in the product. The invention therefore includes a process for the preparation of a compound of the formula IHH

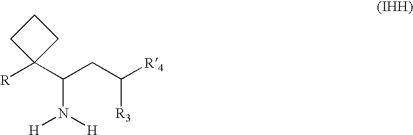

or a salt thereof, where each of R, R₃ and R′₄ are as defined in claim 1, in high enantiomeric purity, which process comprises deacylation a compound of the formula I of high enantiomeric purity, wherein R₁ is H and R₂ is acyl, by treatment with a base or especially by treatment with an acid.

Compounds of formula IHH such as 3, or salts thereof, may be mono-alkylated without racemization following methods known in the art, such as:
1) reaction with chloromethyltriethoxysilane and hydrolysis (see Eur. J. Org. Chem. 2004, 2582);
2) formylation to the formamide and subsequent amide reduction with borane to the monomethylamine (see Tetrahedron Letters 1982, 23, 3315);
3) reaction with dimethyl carbonate in the presence of a zeolith or faujasite (see J. Chem. Soc., Perkin Transactions 1 1997, 1041-1045);
4) alkylation of iminophosphorane with subsequent hydrolysis (see Synthesis 1980, 295);
5) alkylation with trialkyl (especially trimethyl) orthoformate in the presence of sulfuric acid (see: Synth. Commun. 1997, 27, 691-699);
6) caesium hydroxide promoted chemoselective N-alkylation (see Org. Lett. 1999, 1, 1893);
7) transition metal catalysed alkylation with methanol (see J. Chem. Soc. Chem. Commun. 1981, 611-12).

Examples for suitable monoalkylation methods include treatment with a methylating agent, e.g. methyl iodide, dimethyl sulfate and the like, usually in the presence of a suitable base (e.g. a metal hydride such as NaH, or an alkaline hydroxide, especially CsOH) and a suitable solvent, which may be selected from solvents known in the art (see preferred ones further below); more preferred solvents for this reaction include dimethyl formamide (DMF), NMP, dimethylsulfoxide (DMSO), lower alcohols such as ethanol, toluene, ethers such as tetrahydrofuran (THF) etc.

The product may conveniently be isolated as a salt or by distillation.

The invention therefore includes a process for the preparation of a compound of the formula IMeH

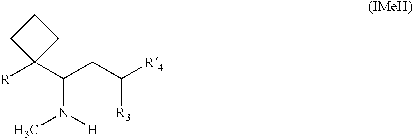

where each of R, R₃ and R′₄ are as defined in claim 1, in high enantiomeric purity, which process comprises methylation of a compound of the formula I of high enantiomeric purity as defined in claim 1, wherein R₁ is H and R₂ is acyl, and subsequent deacylation by treatment with a base or especially by treatment with an acid, or first deacylation of the compound of the formula I wherein R₁ is H and R₂ is acyl, and subsequent monomethylation.

N,N-Dimethylation of an enantiopure compound of formula IHH (such as 3) or a salt thereof may conveniently be effected without racemization by treatment with formic acid and formaldehyde, e.g. following the steps described by Jeffrey et al. in J. Chem. Soc. Perkin Trans. 1 1996, 21, 2583-2590; see especially page 2587. Thus, the invention also pertains to a process for the preparation of a compound of the formula IMeMe

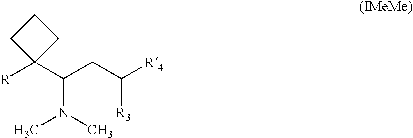

where each of R, R₃ and R′₄ are as defined in claim 1, in high enantiomeric purity, which process comprises deacylation the compound of the formula I wherein R₁ is H and R₂ is acyl by treatment with a base or especially by treatment with an acid, and subsequent treatment with formic acid and formaldehyde.

Usually, the interconversion processes of the invention for preparing the compounds of formulae IHH, IMeH, IMeMe are able to retain the enantiomeric purity of the educt to a high degree; typically, the enantiomeric yield in the present processes is 90% or higher, especially 95% or higher such as >99%.

Whereever used, the term lower alkyl mainly stands for an alkyl group of 1 to 7 carbon atoms, especially for C₁-C₄alkyl; thus, lower alkanols or lower alcohols preferably are C₁-C₁alkanols, especially C₁-C₄alkanols.

Reactions are often carried out under exclusion of oxygen, e.g. by using inert gas such as argon or nitrogen, and under anhydrous conditions, e.g. using the Schlenk technology and equipment, or other methods known in the art.

All reactions are preferably carried out in the presence of a suitable solvent, e.g. an inert organic solvent. Solvents are preferably selected from class 3 solvents (classification by the U.S. food and drug administration); in case of acidic solvents, these may the same time be used for obtaining an acid addition salt. Preferred solvents include water, lower alkyl alcohols, esters, ketones, sulfoxides, ethers, or suitable alkanes, or mixtures of these solvents. Also preferred are DMF, NMP, DMSO, ethanol, methanol, propanol, butanol, toluene, THF, ether.

Reaction temperatures may generally be chosen from ranges convenient for industrial preparations, e.g. from the range between 0° C. and the boiling point of the solvent employed, if any; examples are 0-150° C. or 15-130° C.

The asymmetric hydrogenation of compounds of the formula 11 (e.g. 10 or 8) to give enantiomerically highly enriched or enantiopure compounds of the formula I or VII (e.g. 9) with chiral Ru-catalysts is preferably carried out at temperatures of 20- 120, especially 25-90, most preferably 30-80° C.

The following examples are for illustrative purposes only and are not to be construed to limit the instant invention in any manner whatsoever. Room temperature depicts a temperature in the range 20-25° C. Percentages are by weight unless otherwise indicated.

Abbreviations used in the examples or elsewhere:
¹H-NMR nuclear magnetic resonance (NMR) of ¹H
Ac acetyl
AP area-%
BARF tetrakis(pentafluorophenyl) borate anion
COD 1,5-cyclooctadiene
DCE dichloroethane
DCM dichloromethane
DMAP p-dimethylamino pyridine
ee enantiomeric excess
Et C₂H₅ (=ethyl)
Me CH₃ (=methyl)
NMP

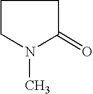

S/C substrate/catalyst ratio
SM starting material
THF tetrahydrofuran
TFA trifluoroacetic acid
TFE trifluoroethanol

### EXAMPLES

### Example 1a (Reference)

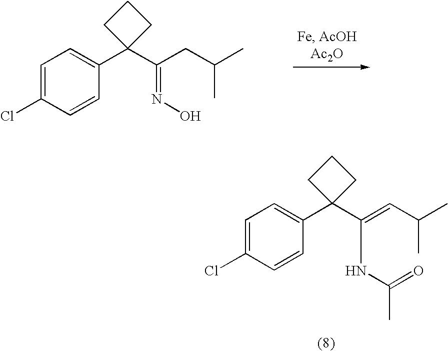

Synthesis of N-{1-[1-(4-Chloro-phenyl)-cyclobutyl]-3-methyl-but-1-enyl}-acetamide 8 via reduction of 1-[1-(4-Chloro-phenyl)-cyclobutyl]-3-methyl-butan-1-one oxime with iron filings:

Preparation of the Oxime: To a solution of 1-[1-(4-Chloro-phenyl)-cyclobutyl]-3-methyl-butan-1-one (14.4 g, 57.6 mmol, prepared as described in J. Chem. Soc. Perkin Trans. 1, 1996, 21, 2583) and hydroxyl ammonium chloride (4.8 g, 69.2 mmol) in ethanol (58 ml), pyridine (5.8 ml) is added and this mixture refluxed for 19 hours. More hydroxyl ammonium chloride (2.0 g, 28 mmol) is added to complete the conversion of the ketone, and the mixture is kept at reflux for another 36 hours. The solvent is then removed on the rotavapor, and the residue is dissolved in ether (500 ml). This solution is washed with 1 N HCl (4 times 80 ml). The organic layer is then dried (sodium sulfate), and removal of the solvent gives 10.0 g of the oxime as a colourless solid (65% yield). ¹H-NMR (CDCl₃, 400 MHz) δ 0.64 (d, 6 H), 1.60-1.92 (m, 2+1 H), 1.95 (d, 2 H), 2.20-2.32 (m, 2 H), 2.50-2.70 (m, 2 H), 7.10-7.25 (m, 4 H). ¹³C-NMR (CDCl₃, 100 MHz) δ 16.8 (CH₂), 23.3 (CH₃); 26.0 (CH), 32.3 (CH₂), 35.6 (CH₂); 52.5 (C), 128.8 (Ar—CH), 128.9 (Ar—CH), 132.5 (Ar—C), 144.1 (Ar—C), 163.1 (C═NOH).

Reduction to the Enamide 8: The oxime (0.5 g, 1.88 mmol) is dissolved in toluene (3.5 ml), and to this solution is added acetic acid (0.32 ml, 5.66 mmol) and acetic anhydride (0.53 ml, 5.64 mmol). Then iron powder (0.21 g, 3.75 mmol, freshly activated by washing with 1 N HCl, washed and dried) is added to this solution, and the rusty brown mixture is stirred at 70° C. for 16 hours. After filtration and removal of the solvent on the rotavapor, the residue is chromatographed on silica to give the product as a crystalline solid (0.18 g, 32%). ¹³C-NMR (CDCl₃, 100 MHz) δ 16.3 (CH₂); 22.8 (2 CH₃); 23.7 (CH₃); 27.9 (CH); 32.9 (2 CH₂); 51.9 (C); 128.1, 128.9 (4 Ar CH); 131.9 (Ar C); 132.4 (C═CH); 134.5 (C═CH); 145.3 (Ar C); 168.7 (C═O).

### Example 1b

### Synthesis 8 of Via Nitrile Alkylation

A dry three-necked 500 ml flask with nitrogen inlet is charged with 1-(4-chloro-phenyl)-cyclobutanecarbonitrile 4 (20.1 g, 105 mmol) and dry toluene (300 ml). The mixture is cooled to 5° C., and then isobutyl magnesium bromide (79 ml of a 2M solution in diethyl ether, 158 mmol) is added within 15 minutes. The reaction mixture is heated to 105° C., and the diethyl ether continuously removed by distillation. The mixture is kept at reflux (105° C.), and after one hour the starting material is consumed completely (TLC). The reaction mixture is then cooled to 5° C., and after the addition of acetic anhydride (32.1 g, 315 mmol) the yellow suspension is stirred at room temperature for another 3 hours. The reaction is quenched with methanol (30 ml), and then neutralized with a saturated sodium hydrogen carbonate solution (200 ml). After the addition of diethyl ether (300 ml) two layers are formed. The organic layer is washed twice with water, and dried over sodium sulfate. Evaporation of the solvent in vacuo gives a yellow-orange solid (35 g), which is recrystallized from hexane to give 8 as pale yellow crystals (19 g, 62%). According to ¹H-NOE experiments, the product is the (Z)-stereoisomer. ¹H NMR (DMSO-D6, 300 MHz) δ 0.88 (d, 6 H. ³J=6.8 Hz, 2 CH₃); 1.60-1.90 m (2 H, CH₂CH₂CH₂); 1.78 (s, 3 H, CH₃); 2.10-2.32 (m, 2 H, CH₂CH₂CH₂); 2.28 (m, 1 H, CH(CH₃)₂); 2.40-2.50 (2 H, CH₂CH₂CH₂); 5.15 (d, 1 H, J=9.6 Hz, C═CH); 7.21, 7.28 (2*2 H, Ar—H); 8.09 (br s, 1 H, NH). ¹³C NMR (DMSO-D6, 75 MHz) 16.58 (CH₂); 23.16 (2 CH₃); 23.59 (CH₃); 27.39 (CH); 32.76 (2 CH₂); 52.13 (C); 128.34, 128.92 (4 Ar CH); 130.83 (Ar C—Cl); 131.31 (C═CH); 136.83 (C═CH); 146.57 (Ar C); 168.46 (C═O).

### Example 2

### Synthesis of (Z)-N-{1-[1-(4-Chloro-phenyl)-cyclobutyl]-3-methyl-buta-1,3-dienyl}-acetamide 10

A dry 500 ml three-necked flask with nitrogen inlet is charged with 1-(4-chloro-phenyl)-cyclobutanecarbonitrile 4 (20.1 g, 105 mmol) and dry THF (300 ml). The mixture is cooled to 5° C., and methallyl magnesium chloride (105 ml of a freshly prepared solution 1.5 M in THF, 158 mmol, 1.5 eq.) is added within 30 minutes. The reaction mixture is stirred for another 30 minutes at 5° C., and then slowly warmed to room temperature, before acetic anhydride (315 ml of a 1 M solution in THF, 315 mmol, 3 eq.) is added. The orange reaction mixture is stirred at 60° C. until the acetylation is complete (2-4 h, monitored by TLC). This gave a mixture, which contained both 10 and the N-di-acetylated product (ca. 1:2 ratio). The excess of acetic anhydride is quenched with 20 ml methanol, and after the addition of sodium methylate (160 g of a 15% solution in methanol, 445 mmol), and further stirring for 15 minutes the di-acetylated 3 had been de-acetylated to give 10. The reaction mixture is then diluted with ethylacetate (250 ml), and washed with saturated ammonium chloride (500 ml), brine (500 ml), and water (500 ml). The organic layer is dried (sodium sulfate), and removal of the solvent in vacuo gave 10 (30 g) as beige crystals. Recrystallization from di-isopropyl ether furnished pure 10 (20 g, 67%) as pale beige crystals. From ¹H-NOE experiments the major stereoisomer is the (Z)-stereoisomer, mp=120° C. ¹H NMR (DMSO-D6, 300 MHz) δ 1.63-1.74 (m, 2 H, CH₂); 1.74 (s (br), 3 H, CH₃); 1.78, (s, 3 H, CH₃C═O); 2.28, 2.49 (2 m, 2 H each, 2 CH₂); 4.81 (s, 1 H, Z-C═CH₂); 4.91 (s, 1 H, E-C═CH₂); 5.95 (s, 1 H, C═CH); 7.26, 7.30 (m, 2 H each, H-aryl); 8.34 (s (br), NH), ¹³C NMR (DMSO-D6, 75 MHz) 16.57 (CH₂); 21.39 (CH₃); 23.53 (CH₃C═O); 33.11 (2 CH₂); 53.33 (C), 117.93 (C═CH₂); 125.77 (C═CH); 128.43, 129.11 (4 Ar CH); 130.99 (Ar C—Cl) 139.80 (NC═C); 141.42 (C═CH₂); 146.35 (Ar C); 169.22 (C═O).

### Example 3

### Hydrogenation Screening of 10 with Achiral Rhodium and Ruthenium Catalysts

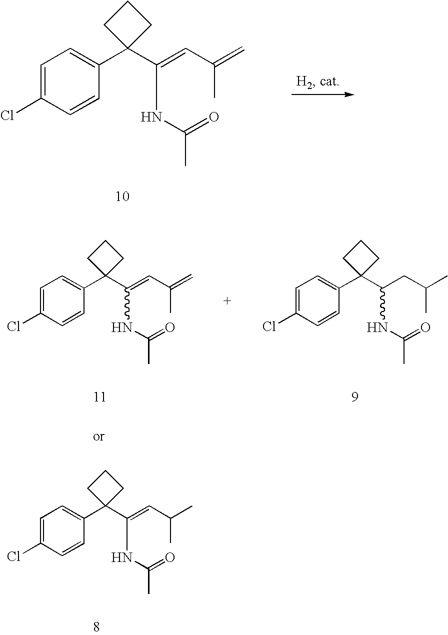

Typical Procedure: To a 10 ml Schlenk flask with a magnetic stirring bar is charged the respective catalyst. The Schlenk flask is evacuated and flushed with argon for 3 times. Then the degassed solvent (3 ml) is added, and the catalyst dissolved. The substrate 10 is transferred into a 25 ml Schlenk flask, which is purged by three cycles vacuum/argon flushing, and then dissolved in the solvent (3 ml). The solution of both the catalyst and the substrate is transferred sequentially into a 50 ml thermostated stainless steel autoclave, which is equipped with a magnetic stirring bar under an argon atmosphere. The autoclave is submitted to hydrogen pressure (10 bar) and the pressure released. After three cycles, the pressure and temperature are set to the desired level, and 20 minutes later magnetic stirring is started. After 17 hours the autoclave is cooled to ambient temperature and the pressure released. The resulting pale yellow solution is evaporated under reduced pressure (rotavapor, max bath=40° C.) to give the product mixture which is analyzed using the assay from Example 4.

Table of results:

| | | | | | | Products | | |
|---|---|---|---|---|---|---|---|---|
| | | | | pH₂ | T | rac. 9 | rac. 11 | 8 |
| Catalyst Precursor | 1. Ligand | S/C | Solvent | [bar] | [° C.] | [AP %] | [AP %] | [AP %] |
| [Rh—(COD)-Dipfc]BF₄ | — | 200 | MeOH | 5 | 25 | 0 | <1% | 100 |
| [Rh—(COD)-Dipfc]BF₄ | — | 50 | MeOH | 10 | 60 | 0 | <1% | 100 |
| [Rh—(NBD)-Cl]₂ | Biphep | 100 | DCE | 20 | 50 | 0 | <1% | 100 |
| [RuCl₂-(p-cymene)]₂ | Biphep | 100 | MeOH | 50 | 50 | 100 | <1% | 0 |

Reaction conditions: 150-200 mg starting material; [10] = 0.15-0.2 M; reaction time 16-17 hrs

### Example 4

### Analytical Assay for the Products

The following HPLC-method is used for the determination of the ee of the products.

| Column: | Chiracel OD-H, Ø = 0.46 cm, length: 25 cm | |
|---|---|---|
| Mobile Phase | Hexane/EtOH 98:2 | |
| Flow: | 0.7 | ml/min |
| Temperature: | 25° | C. |
| Detection | 230 | nm |
| Injection volume: | 1.0 | μl |
| Sample preparation: | ca. 5 mg in 1 ml hexane/ethanol (4:1) | |
| Di-enamide 10 | 23.6 | min |
| Mono-enamide 8 | 17.2 | min |
| Enantiomers of amide 11 | 14.5 and 15.5 min | |
| Amide (S)-9 | 12.9 | min |
| Amide (R)-9 | 13.9 | min |

### Example 5

### Enantioselective Hydrogenation of Monoenamide 8 at S/C≧100

| Precatalyst or | | T | Conv. | ee |
|---|---|---|---|---|
| Precursor + Ligand | S/C | [° C.] | [AP %] | [%] |
| [RuCl₂(p-cymene)]₂ | 100 | 50 | 60 | 85 |
| (R)-MeOBiphep | | | | |
| [RuCl₂(p-cymene)]₂ | 500 | 50 | 68 | 95.0 |
| (R)-MeOBiphep | | | | |
| [RuCl₂(p-cymene)]₂ | 500 | 80 | 60 | 87.4 |
| (R)-MeOBiphep | | | | |
| [RuCl₂(p-cymene)]₂ | 500 | 50 | 63 | 96.4 |
| (R)-BINAP | | | | |
| [RuCl₂(p-cymene)]₂ | 500 | 80 | 42 | 85.9 |
| (R)-BINAP | | | | |
| [RuCl₂(p-cymene)]₂ | 500 | 120 | 63 | 80.6 |
| (R)-BINAP | | | | |

Reaction conditions: metal precursor: [RuCl₂(p-cymene)]₂; 0.25-1.0 g starting material; [8] = 0.15-0.2 M in EtOH; pH₂ = 50 bar; reaction time 16-18 hrs

### Example 6

### Catalyst Screening for the Hydrogenation of 10 with Chiral Ruthenium and Rhodium Catalysts at S/C=100

| Precatalyst or | | pH₂ | T | Conv. of | 8 | 9 | ee 9 |
|---|---|---|---|---|---|---|---|
| Precursor + Ligand | Solv. | [bar] | [° C.] | 10 [AP %] | [AP %] | [AP %] | [%] |
| [RuCl₂(p-cymene)]2 | EtOH | 50 | 50 | 100 | — | 100 | 93.9 |
| (R)-BINAP | | | | | | | |
| [RuCl₂(p-cymene)]₂ | EtOH | 50 | 25 | 67 | 52 | 13 | 98.6 |
| (R)-BINAP | | | | | | | |
| [RuCl₂(p-cymene)]₂ | EtOH | 50 | 80 | 100 | 1 | 98 | 88.5 |
| (R)-BINAP | | | | | | | |
| [RuCl₂(p-cymene)]₂ | EtOH | 10 | 50 | 100 | 23 | 76 | 95.1 |
| (R)-BINAP | | | | | | | |
| [RuCl₂(p-cymene)]₂ | EtOH | 90 | 50 | 100 | 25 | 75 | 92.5 |
| (R)-BINAP | | | | | | | |
| [RuCl₂(p-cymene)]₂ | MeOH | 50 | 50 | 100 | <1 | 98 | 92.7 |
| (R)-BINAP | | | | | | | |
| [RuCl₂(p-cymene)]₂ | EtOH | 50 | 50 | 100 | 48 | 52 | 94.0 |
| (R)-BINAP | | | | | | | |
| RuCl₃•3H₂O | EtOH | 50 | 50 | 100 | 58 | 41 | 84.9 |
| (R)-BINAP | | | | | | | |
| [RuCl₂—(R)-BINAP]₂•Et₃N | EtOH | 50 | 50 | 100 | 15 | 84 | 92.9 |
| [RuCl—(R)-BINAP- | EtOH | 50 | 50 | 100 | 10 | 89 | 91.0 |
| (p-cymene)]Cl | | | | | | | |
| [RuCl₂(p-cymene)]₂ | EtOH | 50 | 50 | 100 | — | 100 | 93.7 |
| (R)-Tol BINAP | | | | | | | |
| [RuCl₂(p-cymene)]₂ | EtOH | 50 | 50 | 100 | — | 100 | 95.4 |
| (R)-Biphemp | | | | | | | |
| [RuCl₂(p-cymene)]₂ | EtOH | 50 | 50 | 100 | — | 100 | 98.3 |
| (R)-MeOBIPHEP | | | | | | | |
| [RuCl₂(p-cymene)]2 | EtOH | 50 | 50 | 100 | — | 100 | 97.6 |
| (R)-MeOBIPHEP | | | | | | | |
| [RuCl₂(p-cymene)]₂ | EtOH | 50 | 50 | 100 | 89 | 11 | 89.1 |
| Taniaphos | | | | | | | |
| [Rh—(COD)-(R,R)- | MeOH | 1 | 25 | 0 | — | — | — |
| EtDuphos]BF₄ | | | | | | | |
| [Rh—(COD)-(R,R)- | MeOH | 20 | 50 | 99^{a} | 66 | 9 | 66 |
| EtDuphos]BF₄ | | | | | | | |

Reaction conditions: 150-200 mg starting material; [10] = 0.15-0.2 M; reaction time 16-68 hrs
a: 25% internally hydrogenated product 11 is deteceted

### Example 7

### Asymmetric Hydrogenation of 10 with Chiral Ruthenium Catalysts at S/C≧500

| Precatalyst or | | pH₂ | T | Conv. of | 8 | 9 | ee 9 |
|---|---|---|---|---|---|---|---|
| Precursor + Ligand | Solvent | [bar] | [° C.] | 10 [AP %] | [AP %] | [AP %] | [%] |
| [RuCl₂(p-cymene)]₂ | EtOH | 50 | 50 | 95 | 64 | 30 | 98.0 |
| (R)-BINAP | | | | | | | |
| [RuCl₂(p-cymene)]₂ | EtOH/DCM | 50 | 50 | 100 | 28 | 72 | 91.9 |
| (R)-BINAP | (4:1) | | | | | | |
| [RuCl₂(p-cymene)]₂ | EtOH/THF | 50 | 50 | 100 | 92 | 8 | 27 |
| (R)-BINAP | (1:4) | | | | | | |
| [RuCl₂(p-cymene)]₂ | TFE | 50 | 50 | 100 | 94 | 6 | 28 |
| (R)-BINAP | | | | | | | |
| [RuCl₂(p-cymene)]₂ | EtOH | 20 | 70 | 100 | 82 | 6 | n.d. |
| (R)-BINAP | | | | | | | |
| [RuCl₂(p-cymene)]₂ | EtOH | 50 | 50 | 100 | 45 | 55 | 96.5 |
| (R)-BINAP | | | | | | | |
| [Rul₂(tripb)]₂ | EtOH | 50 | 50 | 100 | 99 | <1 | n.d. |
| (R)-BINAP | | | | | | | |
| [RuCl₂(p-cymene)]₂ | EtOH | 50 | 50 | 100 | 18 | 82 | 98.6 |
| (R)-MeOBiphep | | | | | | | |
| [RuCl₂(p-cymene)]₂ | EtOH | 50 | 80 | 100 | 0 | 100 | 96.4 |
| (R)-MeOBiphep | | | | | | | |
| [RuCl₂(p-cymene)]₂ | i-PrOH | 50 | 70 | 100 | 58 | 42 | 92.1 |
| (R)-MeOBiphep | | | | | | | |
| [RuCl₂(p-cymene)]₂ | EtOH | 50 | 50 | 97.6 | 49 | 49 | 98.8 |
| (R)-MeOBiphep | | | | | | | |
| [RuCl₂(p-cymene)]₂ | EtOH | 50 | 50 | 100 | 96 | 1 | n.d. |
| (R)-MeOBiphep | | | | | | | |
| [Ru—(R)-MeOBiphep- | EtOH | 50 | 50 | 62 | 25 | 19 | 97 |
| (TFA)₂] | | | | | | | |
| [Ru—(R)-MeOBiphep- | EtOH | 50 | 50 | 12 | 6 | 5 | n.d. |
| (OAc)₂] | | | | | | | |
| [RuCl₂(p-cymene)]₂ | EtOH | 50 | 50 | 100 | 92 | 8 | n.d. |
| (R)-3,4,5-MeO- | | | | | | | |
| TriMeOBiphep | | | | | | | |
| [RuCl₂(p-cymene)]₂ | EtOH | 50 | 50 | 100 | 48 | 52 | 94.0 |
| (R)-Solphos | | | | | | | |
| [RuCl₂(p-cymene)]₂ | EtOH | 50 | 80 | 100 | 5 | 95 | 98.7 |
| (R)-MeOBiphep | | | | | | | |
| [RuCl₂(p-cymene)]₂ | EtOH | 50 | 100 | 100 | 0 | 100 | 95.2 |
| (R)-MeOBiphep | | | | | | | |

### Example 8

### Enantioselective Hydrogenation of 10 at S/C=1500

A 10 ml Schlenck flask equipped with a magnetic stirring bar is charged with [Ru—Cl₂-(p-cymene)]₂ (1.40 mg, 2.3 μmol) and (R)-MeOBiphep (2.80 mg, 4.8 μmol), evacuated under high vacuum/argon flushing (this operation is repeated 3 times) and then EtOH (3 ml) is added with stirring. Dienamide 10 (2.00 g, 6.90 mmol) is taken into a 25 ml Schlenck flask, set under argon and dissolved in ethanol (13 ml). The catalyst and starting material solutions are transferred sequentially to a 50 ml thermostated stainless steel autoclave equipped with a magnetic stirring bar, under argon atmosphere. The autoclave is submitted to hydrogen pressure (10 bar) and the pressure released. After three cycles, the pressure is set to 50 bar and the temperature to 100° C.; 20 minutes later, magnetic stirring is started. After 17 hours, the pressure is released and the resulting pale yellow solution evaporated under reduced pressure (rotavapor, max bath T° C.=40) to give amide 9 in quantitative yield and 95.2% ee (R).

### Example 9

### Hydrogenation of 10 with the Ru-BINAP Catalyst in the Presence of Rate Enhancing Additives

| | | | PH₂ | T | Conv. | 8 | 9 | ee 9 |
|---|---|---|---|---|---|---|---|---|
| S/C | Solvent | Additive | [bar] | [° C.] | [AP %] | [AP %] | [AP %] | [%] |
| 500 | EtOH | — | 50 | 50 | 95 | 64 | 30 | 98.0 |
| 400 | EtOH/TFE | — | 50 | 50 | 100 | ca 70 | ca 30 | >90 |
| 400 | EtOH | HCl 1M | 50 | 80 | 100 | 0 | 100 | 92.0 |
| | | (0.43eq./SM) | | | | | | |
| 1000 | EtOH | HCl 1M | 50 | 50 | 100 | 0 | 100 | 84.7 |
| | | (0.43eq./SM) | | | | | | |

Reaction conditions: 1-2 g starting material; [10] = 0.15-0.2 M; reaction time 16-17 hrs

### Example 10

### Hydrogenation of 10 with the Ru (R) MeOBiphep Catalyst on Large Scale at High S/C Ratio

A 50 ml Schlenk flask equipped with a magnetic stirring bar is charged with [Ru—Cl₂-(p-cymene)]₂ (42.3 mg, 69.0 mol) and (R)-MeOBiphep (80.4 mg, 138 mol), evacuated under high vacuum/argon flushing (this operation is repeated 3 times) and then charged under stirring with dry, degassed EtOH (20 ml). Dienamide 10 (20.0 g, 69.0 mmol) is taken into a 300 ml autoclave and set under argon. The catalyst solution is transferred via canula under argon atmosphere to the autoclave. Then 130 ml of dry, degassed EtOH is transferred to the autoclave and the resulting mixture submitted to hydrogen pressure (10 bar) and the pressure released. After three cycles, the pressure is set to 50 bar and the temperature to 50° C.; 30 minutes later, magnetic stirring is started. After 26 hours, an ¹H-NMR of a reaction aliquot showed complete conversion to the desired product. The pressure is released, the autoclave set under argon and the the pale yellow solution evaporated under reduced pressure (rotavapor, max bath T/° C.=40) to give amide 9 in quantitative yield (19.9 g) and 98.5% ee (R).

### Example 11

### Hydrolysis of Amide 9 to Di-Desmethylsibutramine 3

Hydrolysis: A 86 ml tantalum autoclave equipped with a Teflon stirring bar is charged with 9 (1.0 g, 3.4 mmol, 95.1% ee) and hydrochloric acid (50 ml, 37% in water, 185 mmol). The autoclave is closed and heating at 180° C. is started. After 90 min. the internal temperature has reached 180° C. at a pressure of 44 bar. After 9 hours, the heating is stopped, and the reaction mixture cooled down to room temperature within 11 hours (at that point the internal pressure is 3 bar). The pressure is released, the autoclave opened and the beige reaction mixture taken out. The autoclave is rinsed with distilled water (3×10 ml) and evaporation of the reaction mixture to dryness (rotavapor and high vaccum at 60° C., 1 h) gives 725 mg of 3 (HCl salt) as a beige solid (yield: 77.9%).

Analysis of the ee of 3 Hydrochloride: The determination of the ee of the obtained 3*HCl is done via re-acetylation of the free amine 3 to amide 9:

An aliquot from the above de-acylation reaction mixture (630 mg of crude 3*HCl) is added to an aqueous solution of sodium hydroxide (10 ml, 1M in water, 10 mmol) and the mixture is extracted with dichloromethane (3×10 ml). The organic phase is dried (sodium sulfate), and directly taken into a 100 ml flask with a stirring bar. Acetic acid anhydride (0.40 ml, 4.2 mmol) is added dropwise with stirring at room temperature, followed by DMAP (51 mg, 0.42 mmol). After 3 hours, the reaction mixture is evaporated to dryness. The ee of this material is 95.7%, which is within experimental error identical to the ee of the starting material 3*HCl.

The crude brown solid is then purified by chromatography on silica gel (eluent: dichloromethane/methanol 95:5) to give 580 mg of acetamide 9 as a beige solid. The yield for the sequence hydrolysis/re-acetylation is 67%, corrected for the aliquot which is used for the re-acetylation).

From the hydrolysis product of the amide from Example 10 (98.5% ee (R)) the optical rotation is measured: [α]²⁵_{D}=+3.16 (c=10.065 in CHCl₃).

### Example 12

### Upgrade of the ee of 9 by Recrystallisation

| Amide 9 | ee | di-isoproyl- | recryst. 9 | ee recryst | ee motherl. |
|---|---|---|---|---|---|
| [g] | [%] | ether [ml] | [g]/[%] | [%] | [%] |
| 1.5 | 92.7 | 80 | 1.3/86.6 | 92.2 | nd |
| 2.5 | 96.6 | 130 | 1.8/72.0 | 99.7 | 89.0 |

Comments: The ee of 9 can be enhanced by (re)crystallisation e.g. from di-isopropylether, provided that the ee of the starting material is sufficiently high.

### Example 13

### N-Methylation of Amide 9

To a 250 ml Schlenk flask equipped with a stirring bar is added under an argon atmosphere potassium hydride (2.19 g, ca 10.9 mmol, ca. 20% suspension in oil) and dry THF (50 ml). The mixture is cooled to 0° C. (external) and a solution of acetamide 9 (1.6 g, 5.4 mmol, 98.3% ee (R)) in dry THF is added slowly over 10 minutes. Vigorous evolution of hydrogen is observed immediately. After one hour at 0° C., methyl iodide (2.0 ml, 32.1 mmol) is added within two minutes, and the reaction mixture is then stirred at room temperature over night. The reaction is quenched carefully with water, and when no more hydrogen is evolved, the reaction mixture is evaporated to dryness. The residue is re-dissolved in ethylacetate (ca 150 ml) and this is extracted with water (ca 30 ml) and brine (ca 30 ml). The organic layer is dried (sodium sulfate) and removal of the solvent gives an oil (containing much of the white oil from the potassium hydride). This oil is dissolved in acetonitrile (50 ml) and extracted with heptane (4×10-20 ml). The acetonitrile phase is again evaporated to dryness to give the desired N-methyl acetamide as a beige solid (1.43 g, 85% yield). Two rotamers ca. 2:1, only data of major rotamer provided: ¹³C-NMR (CDCl₃, 100 MHz) δ 14.51 (CH₂CH₂CH₂); 19.78, 22.15, 23.25 (3 CH₃); 30.11 (CHMe₂); 31.00, 33.41 (CH₂CH₂CH₂); 48.60 (C); 54.41 (CHN); 125.83, 127.06 (2 Ar CH); 129.66 (Ar CCl); 143.81 (Ar C); 170.42 (COMe).

### Example 14

### Methylation of (R)-3 to (R)-2

(R)-3 is methylated according to the method given in Tetrahedron Letters 1982, 23, 3315.

### Example 15

### Reductive di-methylation of (R)-3 to (R)-1

(R)-3 is dimethylated according to the method given by James E. Jeffery et al. (J. Chem. Soc. Perkin Trans. 1; 1996; 21; 2583-2590).

### Example 16

### Reductive Methylation of (R)-2 to (R)-1

(R)-2 is methylated according to the method given by James E. Jeffery et al. (J. Chem. Soc. Perkin Trans. 1; 1996; 21; 2583-2590).

### Examples 17 a-c

### Syntheses of Catalyst Precursors (Tetrahedron: Asymmetry 1991, 2, 51)

17a: Synthesis of [Ru-(COD)-(TFA)₂]

To a 50 ml Schlenck flask equipped with a stirring bar is added [Ru-(COD)-(η³-methallyl)₂] (Acros 29578-2500, 1.02 g, 3.192 mmol). The flask is set under argon atmosphere by three cycles high vaccum/argon flushing. Dry diethylether (Fluka 31685, 10 ml) is added with stirring, trifluoroacetic acid (Fluka, 2.45 ml, 6.38 mmol) is added dropwise over 5 minutes and the reaction mixture is stirred for 1.5 hours at 25° C. The orange solution is concentrated to dryness under reduced pressure to give an orange solid. This is stirred with dry diethyl ether (Fluka 31685, 1.5 ml) for 15 minutes at −10° C., decanted, stirred again with diethylether (Fluka 31685, 1.5 ml), decanted again and finally dried under reduced pressure to give [Ru-(COD)-(TFA)₂] as an air sensitive yellow solid (1.21 g, 2.72 mmol, 85% yield).

17b: Synthesis of [Ru-(R)-MeOBiphep-(TFA)₂]

To a 50 ml Schlenck flask equipped with a stirring bar is added [Ru-(COD)-(TFA)₂], 0.72 g, 1.62 mmol) and (R)-MeOBiphep (Roche, >99% ee, 0.94 g, 1.62 mmol). The flask is set under argon atmosphere by three cycles high vaccum/argon flushing. Dry diethylether (Fluka 31685, 10 ml) and dry tetrahydrofurane (Fluka 87371, 3 ml) are added with stirring and the resulting solution stirred for 20 hours at 40° C. (oil bath). The orange solution is concentrated to dryness under reduced pressure to give a brown orange solid. This solid is stirred with dry diethylether (Fluka 31685, 3 ml) for 15 minutes at 15° C., decanted, and the resulting solid washed with dry pentane (Fluka 76878, 3×5 ml) to give [Ru-(R)-MeOBiphep-(TFA)₂] as a brown-orange solid (1.35 g, 1.49 mmol, 92% yield).

17c: Synthesis of [Ru-(R)-MeOBiphep-(OAc)₂]

To a 50 ml Schlenck flask equipped with a stirring bar is added [Ru-(R)-MeOBiphep-(TFA)₂], 1.25 g, 1.36 mmol) and sodium acetate (Merck 6268, 1.12 g, 13.6 mmol). The flask is set under argon atmosphere by three cycles high vacuum/argon flushing. Dry methanol (Fluka 65542, 11 ml) is added with stirring and the resulting suspension stirred for 2 hours at 40° C. (oil bath). The solvent is carefully removed under reduced pressure to give a yellow orange solid. This is extracted with dry dichloromethane (Fluka 66749, 20 ml) and filtered under argon atmosphere in an other 50 ml Schlenck flask (2×2 ml dichloromethane washings). The orange filtrate is concentrated to dryness under high vacuum for 30 minutes and the solid obtained stirred at room temperature with dry ether (Fluka 31685, 3 ml). The supernatant is decanted and the solid washed with dry pentane (Fluka 76878, 2×2 ml) to give [Ru-(R)-MeOBiphep-(OAc)₂] as a yellow solid (1.038 g, 1.30 mmol, 95% yield).

Annex: Ligand Structures

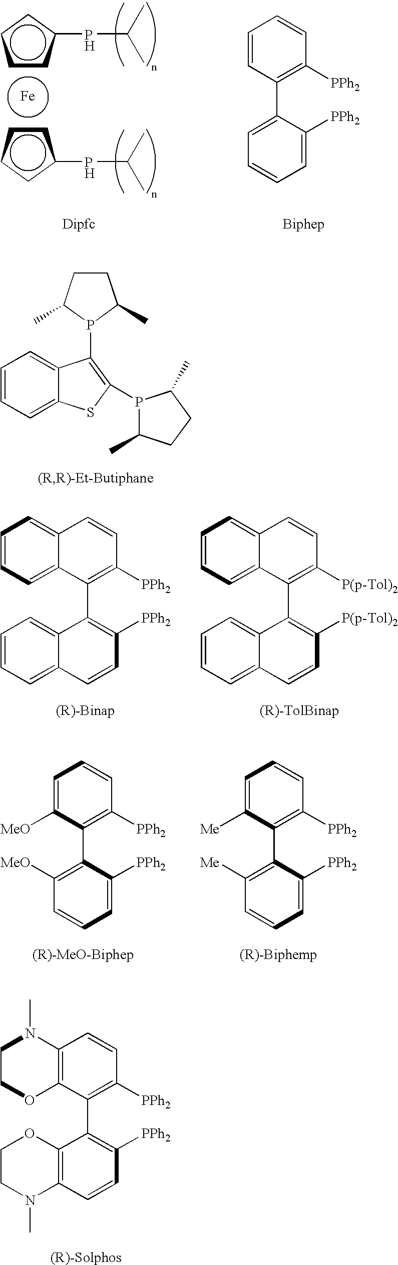

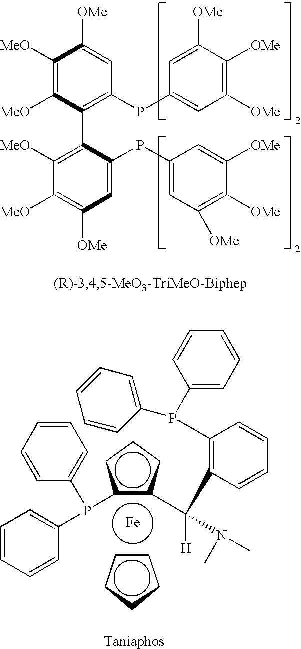

1. A process for the enantioselective preparation of a compound of the formula I 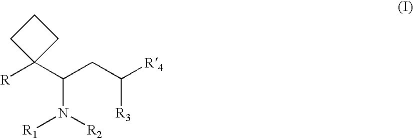
wherein
R is phenyl, or phenyl substituted by Cl, Br, C₁-C₄alkyl or CF₃;
R₁ is H or methyl or ethyl;
R₂ is H or methyl or acyl;
R₃ is H or methyl;
R′₄ is —CH₃ or ═CH₂;
characterized in that a compound of the formula II 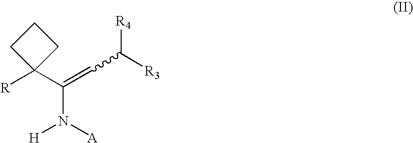
wherein R and R₃ are as in formula I; A is acyl; and R₄ is —CH₃ or ═CH₂;
is hydrogenated in the presence of a chiral Rhodium or Ruthenium catalyst, and a residue R₁ as methyl or ethyl and/or R₂ as H or methyl is subsequently introduced by deacylation and optional alkylation.

2. A process according to claim 1, wherein the compound of formula I is obtained in, or converted into, the form of a pharmaceutically acceptable salt and/or suitable crystalline form.

3. A process according to claim 1, where any acyl in the compound of formulae I and II is C₁-C₄alkanoyl and R₁ is H or methyl.

4. A process according to claim 1, where in the compound of formula I R is 4-chlorophenyl, R₂ is H or methyl and R₃ and R′₄ each is methyl, for the preparation of sibutramine or N-monodesmethyl sibutramine or N,N-didesmethyl sibutramine.

5. A process according to claim 1, wherein the compound of formula I obtained, wherein R₂ is acyl, is crystallized or recrystallized.

6. A process according to claim 1, wherein the ratio of substrate of the formula II to chiral Ruthenium catalyst is greater than 100 and a substance containing a coordinating anion is added.

7. A process according to claim 1, wherein the chiral catalyst is a Ruthenium catalyst containing an axially chiral or planar chiral bisphosphine ligand.

8. A process according to claim 1 wherein the compound of the formula I is obtained with enantiomeric excess of 90% or more of either the (R) or the (S)-enantiomer.

9. A compound of the formula II 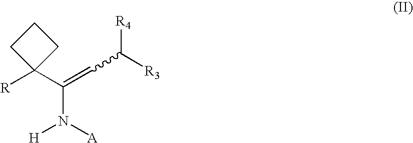
or of the formula VII 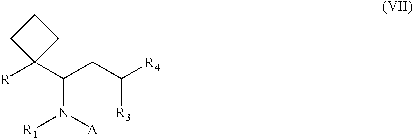
wherein R is phenyl, or phenyl substituted by Cl, Br, C₁-C₄alkyl or CF₃; R₁ is H or methyl; R₃ is H or methyl; R₄ is ═CH₂ or —CH₃; and A is C₁-C₄alkanoyl.

10. A compound according to claim 9 of the formula II or of the formula VII, where the compound of the formula VII is a mixture of the enantiomers with an enantiomeric excess of 90% or more.

11. A process for the preparation of a compound of the formula II 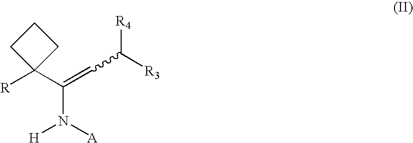
where R is phenyl, or phenyl substituted by Cl, Br, C₁-C₄alkyl or CF₃; R₃ is H or methyl; R₄ is —CH₃ or ═CH₂, and A is acyl; comprising the steps
i) reaction of a nitrile of the formula III 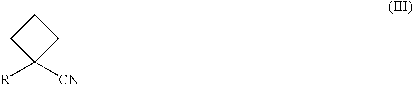
where R is as defined for formula II;
with a reagent of the formula IV
Hal-Mg—CH₂—C(R₃)(R₄) (IV)
where Hal stands for halogen and R₃ and R₄ each are as defined for formula II;
ii) reaction of the metal organic intermediate with an acylating agent introducing the moiety A; and optionally
iii) conversion of a diamide formed into the compound of formula II by reaction with a suitable base.

12. A process according to claim 11, wherein in step
i) the reagent of the formula IV is selected from isobutyl magnesium chloride, isobutyl magnesium bromide, methallyl magnesium chloride or methallyl magnesium bromide; and in
ii) the acylating agent is an acyl halide or anhydride of the formula V or VI
Hal-A (V)
A-O-A (VI)
where any A, independently, is as defined for formula II and Hal is as defined for formula IV.

13. A process according to claim 11, wherein in step
iii) the base is selected from alkoholates and hydroxides of alkali or alkaline earth metals.

14. A process for the preparation of a compound of the formula IHH 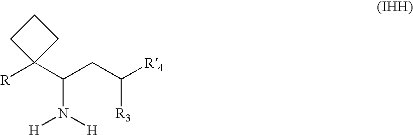
or a salt thereof, where each of R, R₃ and R′₄ are as defined in claim 1, in high enantiomeric purity, which process comprises deacylation a compound of the formula I of high enantiomeric purity as defined in claim 1, wherein R₁ is H and R₂ is acyl, by treatment with a base or by treatment with an acid.

15. A process for the preparation of a compound of the formula IMeH 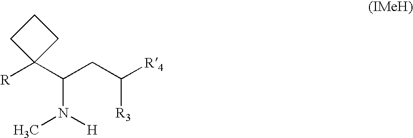
or a salt thereof, where each of R, R₃ and R′₄ are as defined in claim 1, in high enantiomeric purity, which process comprises methylation of a compound of the formula I of high enantiomeric purity as defined in claim 1, wherein R₁ is H and R₂ is acyl, and subsequent deacylation by treatment with a base or by treatment with an acid, or first deacylation of the compound of the formula I wherein R₁ is H and R₂ is acyl, and subsequent monomethylation.

16. A process for the preparation of a compound of the formula IMeMe 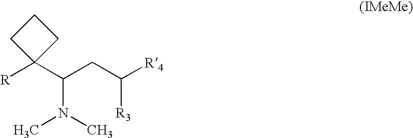
or a salt thereof, where each of R, R₃ and R′₄ are as defined in claim 1, in high enantiomeric purity, which process comprises deacylation the compound of the formula I wherein R₁ is H and R₂ is acyl by treatment with a base or by treatment with an acid, optional monomethylation, and subsequent treatment with formic acid and formaldehyde.

17. A pharmaceutical preparation comprising an effective amount of a compound of the formula II or VII according to claim 9.

18. A process according to claim 3, where any acyl in the compound of formulae I and II is formyl or acetyl and R₁ is H or methyl.

19. A process according to claim 6, wherein the substance containing a coordinating anion is a protic acid or a lithium salt.

20. A compound according to claim 9 of the formula II 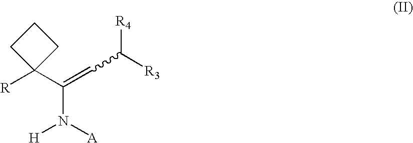
or of the formula VII 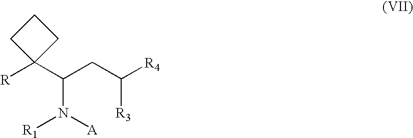
wherein R is 4-chlorophenyl; R₁ is H or methyl; R₃ is H or methyl; R₄ is ═CH₂ or —CH₃; and A is formyl or acetyl.
